(19) United States
(12) Patent Application Publication
(10) Pub. No.: US 2004/0105820 A1
(43) Pub. Date: Jun. 3, 2004

(54) Phospholipid-based powders for inhalation

(76) Inventors: Jeffry G. Weers, Half Moon Bay, CA (US); Thomas E. Tarara, Burlingame, CA (US); Andrew Clark, Woodside, CA (US)

Correspondence Address: NEKTAR THERAPEUTICS, 150 INDUSTRIAL ROAD, SAN CARLOS, CA, 94070, US

(21) Appl. No.: 10/616,448

(22) Filed: Jul. 8, 2003

Related U.S. Application Data
(63) Continuation of application No. 09/888,311, filed on Jun. 22, 2001, now abandoned.
(63) Continuation-in-part of application No. 09/886,296, filed on Jun. 21, 2001.
Application No. 09/886,296 is a division of application No. 09/219,736, filed on Dec. 22, 1998, now Pat. No. 6,565,885.
Application No. 09/219,736 is a continuation of application No. PCT/US98/20602, filed on Sep. 29, 1998.
Application No. PCT/US98/20602 is a continuation-in-part of application No. 09/133,848, filed on Aug. 14, 1998, now abandoned.
Application No. 09/133,848 is a continuation-in-part of application No. 09/106,932, filed on Jun. 29, 1998, now abandoned.
(60) Provisional application No. 60/216,621, filed on Jul. 7, 2000.
(60) Provisional application No. 60/060,337, filed on Sep. 29, 1997.

(51) Int. Cl. A61L 9/04; A61K 9/14

(52) U.S. Cl. 424/46

(57) ABSTRACT

Methods for inhalation are provided. The formulations for inhalation are engineered to be highly dispersible and provide rapid absorption of the active agent so delivered, as well as substantially independent emitted doses and lung deposition as functions of device resistance and inspiratory flow rates, respectively. The present invention also provides reductions in the flow rate dependence in lung deposition and improvements in patient reproducibility.

### RELATED APPLICATIONS

This application claims the priority of U.S. Provisional Application U.S. Provisional Application 60/216,621 filed Jul. 7, 2000.

### FIELD OF THE INVENTION

The present invention relates to particulate compositions and methods for inhalation drug delivery. In particular, the present invention provides phospholipid-containing particulate compositions and methods for pulmonary administration via dry powder inhalers.

### BACKGROUND OF THE INVENTION

This invention relates generally to the field of drug delivery, and in particular to the delivery of pharmaceutical formulations to the lungs. More specifically, the invention relates to the aerosolization of pharmaceutical formulations using energy created by patient inhalation.

Effective drug delivery to a patient is a critical aspect of any successful drug therapy, and a variety of drug delivery techniques have been proposed. For example, one convenient method is the oral delivery of pills, capsules, elixirs and the like. However, oral delivery can in some cases be undesirable in that many drugs are degraded in the digestive tract before they can be absorbed. Another technique is subcutaneous injection. One disadvantage to this approach is low patient acceptance. Other alternative routes of administration that have been proposed include transdermal, intranasal, intrarectal, intravaginal and pulmonary delivery.

Of particular interest to the invention are pulmonary delivery techniques which rely on the inhalation of a pharmaceutical formulation by the patient so that the active drug within the dispersion can reach the distal (alveolar) regions of the lung. A variety of aerosolization systems have been proposed to disperse pharmaceutical formulations. For example, U.S. Pat. Nos. 5,785,049 and 5,740,794, the disclosures of which are herein incorporated by reference, describe exemplary active powder dispersion devices which utilize a compressed gas to aerosolize a powder. Other types of aerosolization systems include MDI's (which typically have a drug that is stored in a propellant), nebulizers (which aerosolize liquids using compressed gas, usually air), and the like.

Another technique which is of interest to the invention is the use of inspired gases to disperse the pharmaceutical formulation. In this way, the patient is able to provide the energy needed to aerosolize the formulation by the patient's own inhalation. This insures that aerosol generation and inhalation are properly synchronized. Utilization of the patient's inspired gases can be challenging in several respects. For example, for some pharmaceutical formulations, such as insulin, it may be desirable to limit the inhalation flow rate within certain limits. For example, PCT/US99/04654, filed Mar. 11, 1999, provides for the pulmonary delivery of insulin at rates less than 17 liters per minute. As another example, copending U.S. patent application Ser. No. 09/414,384 describes pulmonary delivery techniques where a high flow resistance is provided for an initial period followed by a period of lower flow resistance. The complete disclosures of all the above references are herein incorporated by reference.

Another challenge in utilizing the patient's inspired gases is that the inspiration flow rate can drastically vary between individuals. For all commercially available dry powder inhalers, aerosolization and dispersion of the drug formulation are dependent on the inspiratory effort of the patient in inhaling a dose. This effort produces an air flow rate through the device which is governed by the inherent resistance of the device. Variability in inspiratory effort may affect the ability of the formulation to be dispersed within a gas stream, the ability to deagglomerate a powdered formulation, and/or the ability of the aerosolized formulation to adequately reach the deep lung.

Problems associated with variability among patient inspiratory efforts have been addressed through modifications of dry powder inhaler device designs. For example, WO 01/00263 and WO 00/21594, hereby incorporated in their entirety by reference, disclose dry powder inhalers including flow regulation and flow resistance modulation. Examples of other DPIs are disclosed in U.S. Pat. Nos. 4,995,385 and 5,727,546, herein incorporated in their entirety by reference.

Phospholipids are major components of cell and organelle membranes, blood lipoproteins, and lung surfactant. In terms of pulmonary drug delivery, phospholipids have been investigated as therapeutic agents for the treatment of respiratory distress syndrome (i.e. exogenous lung surfactants), and as suitable excipients for the delivery of actives. The interaction of phospholipids with water is critical to the formation, maintenance, and function of each of these important biological complexes (McIntosh and Magid). At low temperatures in the gel phase, the acyl chains are in a conformationally well-ordered state, essentially in the all-trans configuration. At higher temperatures, above the chain melting temperature, this chain order is lost, owing to an increase in gauche conformer content (Seddon and Cevc).

Due to its spreading characteristics on lung epithelia, surfactant has been proposed as the ideal carrier for delivery of drugs to the lung, and via the lung to the systemic circulation. Once again, achieving efficient delivery to the lung is important, especially in light of the potential high cost of many of the current products. One potential way to deliver drugs in phospholipids is as a dry powder aerosolized to the lung. Most fine powders (<5 μm) exhibit poor dispersibility. This can be problematic when attempting to deliver, aerosolize, and/or package the powders.

The major forces that control particle-particle interactions can be divided into short and long range forces. Long-range forces include gravitational attractive forces and electrostatics, where the interaction varies as the square of the separation distance. Short-range attractive forces dominate for dry powders and include van der Waals interactions, hydrogen bonding, and liquid bridging. Liquid bridging occurs when water molecules are able to irreversibly bind particles together.

Examples of particulate compositions incorporating a surfactant are disclosed in PCT publications WO 99/16419, WO 99/38493, WO 99/66903, WO 00/10541, and U.S. Pat. No. 5,855,913, which are hereby incorporated in their entirety by reference.

### SUMMARY OF THE INVENTION

In contrast to the prior art emphasis on device design to address issues commonly associated with patient variability in inspiratory effort, the present invention is directed to a particle engineering approach to overcome such issues. It has surprisingly been found that the particles of the present invention when administered from a simple passive DPI result in an emitted dose and lung deposition that is substantially independent of device resistance and inspiratory effort, respectively. Additionally, it has been discovered that particles of the present invention achieve an unexpectedly more rapid absorption of agent when administered via inhalation.

The present invention provides for dry powder compositions of phospholipid suitable for drug delivery. According to a preferred embodiment, the phospholipid compositions are efficiently delivered to the deep lung. The phospholipid may be delivered alone, as in the case of lung surfactant or in combination with another active agent and/or excipient. According to one embodiment, the compositions of the present invention may be delivered from a simple passive DPI device. The present compositions allow for more efficient delivery to the lung.

It is a further aspect of the present invention that the improvements in dispersibility obtained by the present compositions allow for a simple, passive inhaler device to be utilized, in spite of the fact that particles less than 5 μm are contemplated and generally preferred. Present state-of-the-art formulations for fine particles utilize blends with large lactose particles to improve dispersibility. When placed in a passive DPI device such formulations exhibit a strong dependence of emitted dose and lung deposition on the patient's inspiratory flowrate. The present compositions exhibit little flowrate dependence on the emitted dose and lung deposition.

### BRIEF DESCRIPTION OF THE DRAWINGS

FIG. 1 depicts pharmokinetic profiles of budesonide administered according to the present invention.

FIG. 2 depicts a plot of the in-vitro particle size distribution of a 20% w/w leuprolide acetate PulmoSphere formulation in a multistage liquid impinger operated at various flow rates.

### DEFINITIONS

“Active agent” as described herein includes an agent, drug, compound, composition of matter or mixture thereof which provides some diagnostic, prophylactic, or pharmacologic, often beneficial, effect. This includes foods, food supplements, nutrients, drugs, vaccines, vitamins, and other beneficial agents. As used herein, the terms further include any physiologically or pharmacologically active substance that produces a localized or systemic effect in a patient. The active agent that can be delivered includes antibiotics, antibodies, antiviral agents, antiepileptics, and bronchodilators, and viruses and may be inorganic and organic compounds, including, without limitation, drugs which act on the peripheral nerves, adrenergic receptors, cholinergic receptors, the skeletal muscles, the cardiovascular system, smooth muscles, the blood circulatory system, synaptic sites, neuroeffector junctional sites, endocrine and hormone systems, the immunological system, the reproductive system, the skeletal system, autacoid systems, the alimentary and excretory systems, the histamine system and the central nervous system. Suitable agents may be selected from, for example, polysaccharides, steroids, hypnotics and sedatives, psychic energizers, tranquilizers, anticonvulsants, muscle relaxants, antiParkinson agents, analgesics, anti-inflammatories, muscle contractants, antimicrobials, antimalarials, hormonal agents including contraceptives, sympathomimetics, polypeptides, and proteins capable of eliciting physiological effects, diuretics, lipid regulating agents, antiandrogenic agents, antiparasitics, neoplastics, antineoplastics, hypoglycemics, nutritional agents and supplements, growth supplements, fats, antienteritis agents, electrolytes, vaccines and diagnostic agents.

Examples of active agents useful in this invention include but are not limited to insulin, calcitonin, erythropoietin (EPO), Factor VIII, Factor IX, ceredase, cerezyme, cyclosporine, granulocyte colony stimulating factor (GCSF), alpha-1 proteinase inhibitor, elcatonin, granulocyte macrophage colony stimulating factor (GMCSF), growth hormone, human growth hormone (hGH), growth hormone releasing hormone (GHRH), heparin, low molecular weight heparin (LMWH), interferon alpha, interferon beta, interferon gamma, interleukin-2, luteinizing hormone releasing hormone (LHRH), leuprolide, somatostatin, somatostatin analogs including octreotide, vasopressin analog, follicle stimulating hormone (FSH), immunoglobulins, insulin-like growth factor, insulintropin, interleukin-1 receptor antagonist, interleukin-3, interleukin-4, interleukin-6, macrophage colony stimulating factor (M-CSF), nerve growth factor, parathyroid hormone (PTH), thymosin alpha 1, IIb/IIIa inhibitor, alpha-1 antitrypsin, respiratory syncytial virus antibody, cystic fibrosis transmembrane regulator (CFTR) gene, deoxyribonuclease (DNAse), bactericidal/permeability increasing protein (BPI), anti-CMV antibody, interleukin-1 receptor, 13-cis retinoic acid, nicotine, nicotine bitartrate, gentamicin, ciprofloxacin, amphotericin B, amikacin, tobramycin, pentamidine isethionate, albuterol sulfate, metaproterenol sulfate, beclomethasone dipropionate, triamcinolone acetamide, budesonide, acetonide, ipratropium bromide, flunisolide, fluticasone, fluticasone propionate, salmeterol xinofoate, formoterol fumarate, cromolyn sodium, ergotamine tartrate and the analogues, agonists and antagonists of the above. Active agents may further comprise nucleic acids, present as bare nucleic acid molecules, viral vectors, associated viral particles, nucleic acids associated or incorporated within lipids or a lipid-containing material, plasmid DNA or RNA or other nucleic acid construction of a type suitable for transfection or transformation of cells, particularly cells of the alveolar regions of the lungs. The active agents may be in various forms, such as free base, soluble and insoluble charged or uncharged molecules, components of molecular complexes or pharmacologically acceptable salts. The active agents may be naturally occurring molecules or they may be recombinantly produced, or they may be analogs of the naturally occurring or recombinantly produced active agents with one or more amino acids added or deleted. Further, the active agent may comprise live attenuated or killed viruses suitable for use as vaccines.

As used herein, the term “emitted dose” or “ED” refers to an indication of the delivery of dry powder from a suitable inhaler device after a dispersion event from a powder unit or reservoir. ED is defined as the ratio of the dose delivered by an inhaler device (described in detail below) to the nominal dose (i.e., the mass of powder per unit dose placed into a suitable inhaler device prior to dispersion). The ED is an experimentally-determined amount, and is typically determined using an in-vitro device set up which mimics patient dosing. To determine an ED value, a nominal dose of dry powder (as defined above) is placed into a suitable dry powder inhaler, which is then actuated, dispersing the powder. The resulting aerosol cloud is then drawn by vacuum from the device, where it is captured on a tared filter attached to the device mouthpiece. The amount of powder that reaches the filter constitutes the emitted dose. For example, for a 5 mg, dry powder-containing blister pack placed into an inhalation device, if dispersion of the powder results in the recovery of 4 mg of powder on a tared filter as described above, then the ED for the dry powder composition is: 4 mg (delivered dose)/5 mg (nominal dose)×100=80%.

“Mass median diameter” or “MMD” is a measure of particle size, since the powders of the invention are generally polydisperse (i.e., consist of a range of particle sizes). MMD values as reported herein are determined by laser diffraction.

“Mass median aerodynamic diameter” or “MMAD” is a measure of the aerodynamic size of a dispersed particle. The aerodynamic diameter is used to describe an aerosolized powder in terms of its settling behavior, and is the diameter of a unit density sphere having the same settling velocity, generally in air, as the particle. The aerodynamic diameter encompasses particle shape, density and physical size of a particle. Techniques for measuring MMAD are set forth in the Examples that follow.

As used herein, “passive dry powder inhaler” refers to an inhalation device which relies upon the patient's inspiratory effort to disperse and aerosolize a drug formulation contained within the device and does not include inhaler devices which comprise a means for providing energy to disperse and aerosolize the drug formulation, such as pressurized gas and vibrating or rotating elements.

As used herein, “FPF_{3.3 μm}” refers to the fraction of particles emitted from the passive DPI device with an MMAD of 3.3 μm and below as determined by Anderson Cascade Impaction (ACI) or multi-stage liquid impinger (MSLI).

As used herein, “FPF_{4+F}” refers to the fraction of fine particles depositing on stage 4 and the filter in the MSLI, independent of flow rate. This is analogous to a patient inhaling at different inspiratory rates into a constant lung architecture. The particle stopping distance and hence the deposition profile will change depending on inhalation flow rate Q. Hence FPF_{4+F}, provides a measure of the flow rate dependence of a test aerosol formulation.

### DETAILED DESCRIPTION OF THE INVENTION

The present invention is directed to phospholipid-containing, dispersible particulate compositions and methods for pulmonary administration to the respiratory tract for local or systemic therapy via aerosolization. The invention is based, at least in part, on the surprising discovery of the beneficial aerosolization of phospholipid -containing particulate compositions. These unexpected benefits include rapid absorption of the active agent so delivered, as well as substantially independent emitted doses and lung deposition as functions of device resistance and inspiratory flow rates, respectively. Reductions in the flow rate dependence in lung deposition and improvements in patient reproducibility are especially important for drugs which have a narrow therapeutic index, or for which the dose of drug must be accurately controlled (e.g., a diabetic patient).

According to one embodiment directed to rapid absorption of active agent, it has been surprisingly discovered that particles of the present invention incorporating hydrophobic active agents are rapidly absorbed. According to this embodiment, the pulmonary administration of such agents result in a Tmax within 60 minutes of administration, preferably within 15 minutes of administration. One particularly preferred embodiment is directed to achieving a Tmax within 10 minutes of inhalation.

According to another embodiment, it has surprisingly been discovered that the particles of the present invention when administered via passive dry powder inhalers exhibit an emitted dose substantially independent of device resistance and lung deposition substantially independent of inhalation flow rate. According to a preferred embodiment, methods according to the present invention provide an emitted dose of at least 60% most preferably greater than 80% when administered from a passive dry powder inhaler having a resistance <0.60 (cmH₂O) ^{1/2}/L·min⁻¹ preferably within 0.01-0.30 (cmH₂O)^{1/2}/L·min⁻¹, while also providing lung deposition of at least 20%, preferably at least 25%, which is substantially independent of inhalation flow rates of <90 L/min, preferably 10-60 L/min, and most preferably 12-45 L/min.

While not wishing to be bound to any particular theory, it is believed that the improvements in powder dispersibility are the result of several formulation and process factors:

(a) Low particle density

(b) Decreased interparticle coordination numbers relative to falt micronized crystals, resulting from the spherical particle shape and relatively monodisperse distribution of particle sizes.

(c) Decreased interparticle contact points resulting from spherical shape, and the particle morphology, where contact points may be particle on a pore.

(d) Increased interparticle separation distances resulting from particle morphologies with increased surface roughness.

(e) Decreased interparticle contact areas resulting from the three-dimensional foam-like structure of the particle wall, which provides mechanical strength to resist particle deformation.

In a broad sense, phospholipid suitable for use in the present invention include any of those known in the art. According to a preferred embodiment, the phospholipid is most preferably a saturated phospholipid. According to a particularly preferred embodiment, saturated phosphatidylcholines are used as the phospholipid of the present invention. Preferred acyl chain lengths are 16:0 and 18:0 (i.e. palmitoyl and stearoyl). According to one embodiment directed to lung surfactant compositions, the phospholipid can make up to 90 to 99.9% w/w of the composition. Suitable phospholipids according to this aspect of the invention include natural or synthetic lung surfactants such as those commercially available under the trademarks ExoSurf, InfaSurf® (Ony, Inc.), Survanta, CuroSurf, and ALEC. For drug delivery purposes wherein an active agent is included with the particulate composition, the phospholipid content will be determined by the drug activity, the mode of delivery, and other factors and will likely be in the range from about 10% to up to 99.9% w/w. Thus, drug loading can vary between about 0.1% and 90% w/w, preferably 2-80% w/w.

Phospholipids from both natural and synthetic sources are compatible with the present invention and may be used in varying concentrations to form the structural matrix. Generally compatible phospholipids comprise those that have a gel to liquid crystal phase transition greater than about 40° C. Preferably the incorporated phospholipids are relatively long chain (i.e. C₁₆-C₂₂) saturated lipids and more preferably comprise saturated phospholipids, most preferably saturated phosphatidylcholines having acyl chain lengths of 16:0 or 18:0 (palmitoyl and stearoyl). Exemplary phospholipids useful in the disclosed stabilized preparations comprise, phosphoglycerides such as dipalmitoylphosphatidylcholine, disteroylphosphatidylcholine, diarachidoylphosphatidylcholine dibehenoylphosphatidylcholine, diphosphatidyl glycerol, short-chain phosphatidylcholines, long-chain saturated phosphatidylethanolamines, long-chain saturated phosphatidylserines, long-chain saturated phosphatidylglycerols, long-chain saturated phosphatidylinositols.

In addition to the phospholipid, a co-surfactant or combinations of surfactants, including the use of one or more in the liquid phase and one or more associated with the particulate compositions are contemplated as being within the scope of the invention. By “associated with or comprise” it is meant that the particulate compositions may incorporate, adsorb, absorb, be coated with or be formed by the surfactant. Surfactants include fluorinated and nonfluorinated compounds and are selected from the group consisting of saturated and unsaturated lipids, nonionic detergents, nonionic block copolymers, ionic surfactants and combinations thereof. In those embodiments comprising stabilized dispersions, such nonfluorinated surfactants will preferably be relatively insoluble in the suspension medium. It should be emphasized that, in addition to the aforementioned surfactants, suitable fluorinated surfactants are compatible with the teachings herein and may be used to provide the desired preparations.

Compatible nonionic detergents suitable as co-surfactants comprise: sorbitan esters including sorbitan trioleate (Span™ 85), sorbitan sesquioleate, sorbitan monooleate, sorbitan monolaurate, polyoxyethylene (20) sorbitan monolaurate, and polyoxyethylene (20) sorbitan monooleate, oleyl polyoxyethylene (2) ether, stearyl polyoxyethylene (2) ether, lauryl polyoxyethylene (4) ether, glycerol esters, and sucrose esters. Other suitable nonionic detergents can be easily identified using McCutcheon's Emulsifiers and Detergents (McPublishing Co., Glen Rock, N.J.) which is incorporated herein in its entirety. Preferred block copolymers include diblock and triblock copolymers of polyoxyethylene and polyoxypropylene, including poloxamer 188 (Pluronic™ F-68), poloxamer 407 (Pluronic™ F-127), and poloxamer 338. Ionic surfactants such as sodium sulfosuccinate, and fatty acid soaps may also be utilized.

Other lipids including glycolipids, ganglioside GM1, sphingomyelin, phosphatidic acid, cardiolipin; lipids bearing polymer chains such as polyethylene glycol, chitin, hyaluronic acid, or polyvinylpyrrolidone; lipids bearing sulfonated mono-, di-, and polysaccharides; fatty acids such as palmitic acid, stearic acid, and oleic acid; cholesterol, cholesterol esters, and cholesterol hemisuccinate may also be used in accordance with the teachings of this invention.

It will further be appreciated that the particulate compositions according to the invention may, if desired, contain a combination of two or more active ingredients. The agents may be provided in combination in a single species of particulate composition or individually in separate species of particulate compositions. For example, two or more active agents may be incorporated in a single feed stock preparation and spray dried to provide a single particulate composition species comprising a plurality of active agents. Conversely, the individual actives could be added to separate stocks and spray dried separately to provide a plurality of particulate composition species with different compositions. These individual species could be added to the suspension medium or dry powder dispensing compartment in any desired proportion and placed in the aerosol delivery system as described below. Further, as alluded to above, the particulate compositions (with or without an associated agent) may be combined with one or more conventional (e.g. a micronized drug) active or bioactive agents to provide the desired dispersion stability or powder dispersibility.

Based on the foregoing, it will be appreciated by those skilled in the art that a wide variety of active agents may be incorporated in the disclosed particulate compositions. Accordingly, the list of preferred active agents above is exemplary only and not intended to be limiting. It will also be appreciated by those skilled in the art that the proper amount of agent and the timing of the dosages may be determined for the particulate compositions in accordance with already existing information and without undue experimentation. Preferred agents according to this invention include leuprolide acetate, budesonide, tobramycin sulfate, and PTH.

In addition to the phospholipid, the microparticles of the present invention may also include a biocompatible, preferably biodegradable polymer, copolymer, or blend or other combination thereof. In this respect useful polymers comprise polylactides, polylactide-glycolides, cyclodextrins, polyacrylates, methylcellulose, carboxymethylcellulose, polyvinyl alcohols, polyanhydrides, polylactams, polyvinyl pyrrolidones, polysaccharides (dextrans, starches, chitin, chitosan, etc.), hyaluronic acid, proteins, (albumin, collagen, gelatin, etc.). Examples of polymeric resins that would be useful for the preparation of perforated ink microparticles include: styrene-butadiene, styrene-isoprene, styrene-acrylonitrile, ethylene-vinyl acetate, ethylene-acrylate, ethylene-acrylic acid, ethylene-methylacrylatate, ethylene-ethyl acrylate, vinyl-methyl methacrylate, acrylic acid-methyl methacrylate, and vinyl chloride-vinyl acetate. Those skilled in the art will appreciate that, by selecting the appropriate polymers, the delivery efficiency of the particulate compositions and/or the stability of the dispersions may be tailored to optimize the effectiveness of the active or agent.

Besides the aforementioned polymer materials and surfactants, it may be desirable to add other excipients to a particulate composition to improve particle rigidity, production yield, emitted dose and deposition, shelf-life and patient acceptance. Such optional excipients include, but are not limited to: coloring agents, taste masking agents, buffers, hygroscopic agents, antioxidants, and chemical stabilizers. Further, various excipients may be incorporated in, or added to, the particulate matrix to provide structure and form to the particulate compositions (i.e. microspheres such as latex particles). In this regard it will be appreciated that the rigidifying components can be removed using a post-production technique such as selective solvent extraction.

Other excipients may include, but are not limited to, carbohydrates including monosaccharides, disaccharides and polysaccharides. For example, monosaccharides such as dextrose (anhydrous and monohydrate), galactose, mannitol, D-mannose, sorbitol, sorbose and the like; disaccharides such as lactose, maltose, sucrose, trehalose, and the like; trisaccharides such as raffinose and the like; and other carbohydrates such as starches (hydroxyethylstarch), cyclodextrins and maltodextrins. Other excipients suitable for use with the present invention, including amino acids, are known in the art such as those disclosed in WO 95/31479, WO 96/32096, and WO 96/32149. Mixtures of carbohydrates and amino acids are further held to be within the scope of the present invention. The inclusion of both inorganic (e.g. sodium chloride, etc.), organic acids and their salts (e.g. carboxylic acids and their salts such as sodium citrate, sodium ascorbate, magnesium gluconate, sodium gluconate, tromethamine hydrochloride, etc.) and buffers is also contemplated. The inclusion of salts and organic solids such as ammonium carbonate, ammonium acetate, ammonium chloride or camphor are also contemplated. According to a preferred embodiment, a metal cation, preferably calcium is added to the feed stock from which the particles are prepared as disclosed in U.S. provisional patent application 60/216,621, previously incorporated by reference.

Yet other preferred embodiments include particulate compositions that may comprise, or may be coated with, charged species that prolong residence time at the point of contact or enhance penetration through mucosae. For example, anionic charges are known to favor mucoadhesion while cationic charges may be used to associate the formed microparticulate with negatively charged bioactive agents such as genetic material. The charges may be imparted through the association or incorporation of polyanionic or polycationic materials such as polyacrylic acids, polylysine, polylactic acid and chitosan.

The medicament is formulated in a way such that it readily disperses into discrete particles with an MMD between 0.5 to 20 μm, preferably 0.5-5 μm, and are further characterized by an aerosol particle size distribution less than about 10 μm mass median aerodynamic diameter (MMAD), and preferably less than 5.0 μm. The mass median aerodynamic diameters of the powders will characteristically range from about 0.5-10 μm, preferably from about 0.5-5.0 μm MMAD, more preferably from about 1.0-4.0 μm MMAD.

The administration methods of the present invention utilize passive DPIs. Examples of passive DPIs suitable for administration of the particulate compositions of the present invention are disclosed in U.S. Pat. Nos. 5,673,686, and 4,995,385 and PCT application nos. 00/72904, 00/21594, and 01/00263, hereby incorporated in their entirety by reference. DPI formulations are typically packaged in single dose units such as those disclosed in the above mentioned patents or they employ reservoir systems capable of metering multiple doses with manual transfer of the dose to the device.

Particularly preferred embodiments of the invention incorporate spray dried, hollow and porous particulate compositions as disclosed in WO 99/16419, hereby incorporated in its entirety by reference. Such particulate compositions comprise particles having a relatively thin porous wall defining a large internal void, although, other void containing or perforated structures are contemplated as well. In preferred embodiments the particulate compositions will further comprise an active agent.

Compositions according to the present invention typically yield powders with bulk densities less than 0.5 g/cm³ or 0.3 g/cm³, preferably less 0.1 g/cm³ and most preferably less than 0.05 g/cm³. By providing particles with very low bulk density, the minimum powder mass that can be filled into a unit dose container is reduced, which eliminates the need for carrier particles. That is, the relatively low density of the powders of the present invention provides for the reproducible administration of relatively low dose pharmaceutical compounds. Moreover, the elimination of carrier particles will potentially minimize throat deposition and any “gag” effect, since the large lactose particles will impact the throat and upper airways due to their size.

It will be appreciated that the particulate compositions disclosed herein comprise a structural matrix that exhibits, defines or comprises voids, pores, defects, hollows, spaces, interstitial spaces, apertures, perforations or holes. The absolute shape (as opposed to the morphology) of the perforated microstructure is generally not critical and any overall configuration that provides the desired characteristics is contemplated as being within the scope of the invention. Accordingly, preferred embodiments can comprise approximately microspherical shapes. However, collapsed, deformed or fractured particulates are also compatible.

In accordance with the teachings herein the particulate compositions will preferably be provided in a “dry” state. That is the microparticles will possess a moisture content that allows the powder to remain chemically and physically stable during storage at ambient temperature and easily dispersible. As such, the moisture content of the microparticles is typically less than 6% by weight, and preferably less 3% by weight. In some instances the moisture content will be as low as 1% by weight. Of course it will be appreciated that the moisture content is, at least in part, dictated by the formulation and is controlled by the process conditions employed, e.g., inlet temperature, feed concentration, pump rate, and blowing agent type, concentration and post drying.

Reduction in bound water leads to significant improvements in the dispersibility and flowability of phospholipid based powders, leading to the potential for highly efficient delivery of powdered lung surfactants or particulate composition comprising active agent dispersed in the phospholipid. The improved dispersibility allows simple passive DPI devices to be used to effectively deliver these powders.

As seen from the passages above, various components may be associated with, or incorporated in the particulate compositions of the present invention. Similarly, several techniques may be used to provide particulates having the desired morphology (e.g. a perforated or hollow/porous configuration), dispersibility and density. Among other methods, particulate compositions compatible with the instant invention may be formed by techniques including spray drying, vacuum drying, solvent extraction, emulsification or lyophilization, and combinations thereof. It will further be appreciated that the basic concepts of many of these techniques are well known in the prior art and would not, in view of the teachings herein, require undue experimentation to adapt them so as to provide the desired particulate compositions.

While several procedures are generally compatible with the present invention, particularly preferred embodiments typically comprise particulate compositions formed by spray drying. As is well known, spray drying is a one-step process that converts a liquid feed to a dried particulate form. With respect to pharmaceutical applications, it will be appreciated that spray drying has been used to provide powdered material for various administrative routes including inhalation. See, for example, M. Sacchetti and M. M. Van Oort in: Inhalation Aerosols: Physical and Biological Basis for Therapy, A. J. Hickey, ed. Marcel Dekkar, New York, 1996, which is incorporated herein by reference.

In general, spray drying consists of bringing together a highly dispersed liquid, and a sufficient volume of hot air to produce evaporation and drying of the liquid droplets. The preparation to be spray dried or feed (or feed stock) can be any solution, course suspension, slurry, colloidal dispersion, or paste that may be atomized using the selected spray drying apparatus. In preferred embodiments the feed stock will comprise a colloidal system such as an emulsion, reverse emulsion, microemulsion, multiple emulsion, particulate dispersion, or slurry. Typically the feed is sprayed into a current of warm filtered air that evaporates the solvent and conveys the dried product to a collector. The spent air is then exhausted with the solvent. Those skilled in the art will appreciate that several different types of apparatus may be used to provide the desired product. For example, commercial spray dryers manufactured by Buchi Ltd. or Niro Corp. will effectively produce particles of desired size.

It will further be appreciated that these spray dryers, and specifically their atomizers, may be modified or customized for specialized applications, i.e. the simultaneous spraying of two solutions using a double nozzle technique. More specifically, a water-in-oil emulsion can be atomized from one nozzle and a solution containing an anti-adherent such as mannitol can be co-atomized from a second nozzle. In other cases it may be desirable to push the feed solution though a custom designed nozzle using a high pressure liquid chromatography (HPLC) pump. Provided that microstructures comprising the correct morphology and/or composition are produced the choice of apparatus is not critical and would be apparent to the skilled artisan in view of the teachings herein. Examples of spray drying methods and systems suitable for making the dry powders of the present invention are disclosed in WO 99/16419 previously incorporated by reference and in U.S. Pat. Nos. 6,077,543, 6,051,256, 6,001,336, 5,985,248, and 5,976,574, hereby incorporated in their entirety by reference.

While the resulting spray-dried powdered particles typically are approximately spherical in shape, nearly uniform in size and frequently are hollow, there may be some degree of irregularity in shape depending upon the incorporated medicament and the spray drying conditions. In many instances dispersion stability and dispersibility of the particulate compositions appears to be improved if an inflating agent (or blowing agent) is used in their production as disclosed in WO 99/16419 cited above. Particularly preferred embodiments comprise an emulsion with the inflating agent as the disperse or continuous phase. The inflating agent is preferably dispersed with a surfactant solution, using, for instance, a commercially available microfluidizer at a pressure of about 5000 to 15,000 psi. This process forms an emulsion, preferably stabilized by an incorporated surfactant, typically comprising submicron droplets of water immiscible blowing agent dispersed in an aqueous continuous phase. The formation of such emulsions using this and other techniques are common and well known to those in the art. The blowing agent is preferably a fluorinated compound (e.g. perfluorohexane, perfluorooctyl bromide, perfluorooctyl ethane, perfluorodecalin, perfluorobutyl ethane) which vaporizes during the spray-drying process, leaving behind generally hollow, porous aerodynamically light microspheres. Other suitable liquid blowing agents include nonfluorinated oils, chloroform, Freons, ethyl acetate, alcohols and hydrocarbons. Nitrogen and carbon dioxide gases are also contemplated as a suitable blowing agent. Perfluorooctyl ethane is particularly preferred according to the invention.

Besides the aforementioned compounds, inorganic and organic substances which can be removed under reduced pressure by sublimation in a post-production step are also compatible with the instant invention. These sublimating compounds can be dissolved or dispersed as micronized crystals in the spray drying feed solution and include ammonium carbonate and camphor. Other compounds compatible with the present invention comprise rigidifying solid structures which can be dispersed in the feed solution or prepared in-situ. These structures are then extracted after the initial particle generation using a post-production solvent extraction step. For example, latex particles can be dispersed and subsequently dried with other wall forming compounds, followed by extraction with a suitable solvent.

Although the particulate compositions are preferably formed using a blowing agent as described above, it will be appreciated that, in some instances, no additional blowing agent is required and an aqueous dispersion of the medicament and/or excipients and surfactant(s) are spray dried directly. In such cases, the formulation may be amenable to process conditions (e.g., elevated temperatures) that may lead to the formation of hollow, relatively porous microparticles. Moreover, the medicament may possess special physicochemical properties (e.g., high crystallinity, elevated melting temperature, surface activity, etc.) that makes it particularly suitable for use in such techniques.

Regardless of which blowing agent is ultimately selected, it has been found that compatible particulate compositions may be produced particularly efficiently using a Buichi mini spray drier (model B-191, Switzerland). As will be appreciated by those skilled in the art, the inlet temperature and the outlet temperature of the spray drier are not critical but will be of such a level to provide the desired particle size and to result in a product that has the desired activity of the medicament. In this regard, the inlet and outlet temperatures are adjusted depending on the melting characteristics of the formulation components and the composition of the feed stock. The inlet temperature may thus be between 60° C. and 170° C., with the outlet temperatures of about 40° C. to 120° C. depending on the composition of the feed and the desired particulate characteristics. Preferably these temperatures will be from 90° C. to 120° C. for the inlet and from 60° C. to 90° C. for the outlet. The flow rate which is used in the spray drying equipment will generally be about 3 ml per minute to about 15 ml per minute. The atomizer air flow rate will vary between values of 25 liters per minute to about 50 liters per minute. Commercially available spray dryers are well known to those in the art, and suitable settings for any particular dispersion can be readily determined through standard empirical testing, with due reference to the examples that follow. Of course, the conditions may be adjusted so as to preserve biological activity in larger molecules such as proteins or peptides.

Whatever components are selected, the first step in particulate production typically comprises feed stock preparation. If the phospholipid based particle is intended to act as a carrier for another active agent, the selected active agent is dissolved in a solvent, preferably water, to produce a concentrated solution. A polyvalent cation may be added to the active agent solution or may be added to the phospholipid emulsion as discussed in 60/216,621 previously cited. The active agent may also be dispersed directly in the emulsion, particularly in the case of water insoluble agents. Alternatively, the active agent may be incorporated in the form of a solid particulate dispersion. The concentration of the active agent used is dependent on the amount of agent required in the final powder and the performance of the delivery device employed (e.g., the fine particle dose for a MDI or DPI). As needed, cosurfactants such as poloxamer 188 or span 80 may be dispersed into this annex solution. Additionally, excipients such as sugars and starches can also be added.

In selected embodiments a polyvalent cation-containing oil-in-water emulsion is then formed in a separate vessel. The oil employed is preferably a fluorocarbon (e.g., perfluorooctyl bromide, perfluorooctyl ethane, perfluorodecalin) which is emulsified with a phospholipid. For example, polyvalent cation and phospholipid may be homogenized in hot distilled water (e.g., 60° C.) using a suitable high shear mechanical mixer (e.g., Ultra-Turrax model T-25 mixer) at 8000 rpm for 2 to 5 minutes. Typically 5 to 25 g of fluorocarbon is added dropwise to the dispersed surfactant solution while mixing. The resulting polyvalent cation-containing perfluorocarbon in water emulsion is then processed using a high pressure homogenizer to reduce the particle size. Typically the emulsion is processed at 12,000 to 18,000 psi, 5 discrete passes and kept at 50 to 80° C.

The active agent solution and perfluorocarbon emulsion are then combined and fed into the spray dryer. Typically the two preparations will be miscible as the emulsion will preferably comprise an aqueous continuous phase. While the bioactive agent is solubilized separately for the purposes of the instant discussion it will be appreciated that, in other embodiments, the active agent may be solubilized (or dispersed) directly in the emulsion. In such cases, the active emulsion is simply spray dried without combining a separate active agent preparation.

In any event, operating conditions such as inlet and outlet temperature, feed rate, atomization pressure, flow rate of the drying air, and nozzle configuration can be adjusted in accordance with the manufacturer's guidelines in order to produce the required particle size, and production yield of the resulting dry particles. Exemplary settings are as follows: an air inlet temperature between 60° C. and 170° C.; an air outlet between 40° C. to 120° C.; a feed rate between 3 ml to about 15 ml per minute; and an aspiration air flow of 300 L/min. and an atomization air flow rate between 25 to 50 L/min. The selection of appropriate apparatus and processing conditions are well within the purview of a skilled artisan in view of the teachings herein and may be accomplished without undue experimentation. In any event, the use of these and substantially equivalent methods provide for the formation of hollow porous aerodynamically light microparticles with particle diameters appropriate for aerosol deposition into the lung. In especially preferred embodiments the particulate compositions comprise hollow, porous spray dried microparticles.

Along with spray drying, particulate compositions useful in the present invention may be formed by lyophilization. Those skilled in the art will appreciate that lyophilization is a freeze-drying process in which water is sublimed from the composition after it is frozen. The particular advantage associated with the lyophilization process is that biologicals and pharmaceuticals that are relatively unstable in an aqueous solution can be dried without elevated temperatures (thereby eliminating the adverse thermal effects), and then stored in a dry state where there are few stability problems. With respect to the instant invention such techniques are particularly compatible with the incorporation of peptides, proteins, genetic material and other natural and synthetic macromolecules in particulate compositions without compromising physiological activity. Methods for providing lyophilized particulates are known to those of skill in the art and it would clearly not require undue experimentation to provide dispersion compatible microparticles in accordance with the teachings herein. The lyophilized cake containing a fine foam-like structure can be micronized using techniques known in the art to provide 3 to 10 μm sized particles. Accordingly, to the extent that lyophilization processes may be used to provide microparticles having the desired porosity and size they are in conformance with the teachings herein and are expressly contemplated as being within the scope of the instant invention.

Besides the aforementioned techniques, the particulate compositions or particles of the present invention may also be formed using a method where a feed solution (either emulsion or aqueous) containing wall forming agents is rapidly added to a reservoir of heated oil (e.g. perflubron or other high boiling FCs) under reduced pressure. The water and volatile solvents of the feed solution rapidly boils and are evaporated. This process provides a perforated structure from the wall forming agents similar to puffed rice or popcorn. Preferably the wall forming agents are insoluble in the heated oil. The resulting particles can then separated from the heated oil using a filtering technique and subsequently dried under vacuum.

Additionally, the particulate compositions of the present invention may also be formed using a double emulsion method. In the double emulsion method the medicament is first dispersed in a polymer dissolved in an organic solvent (e.g. methylene chloride, ethyl acetate) by sonication or homogenization. This primary emulsion is then stabilized by forming a multiple emulsion in a continuous aqueous phase containing an emulsifier such as polyvinylalcohol. Evaporation or extraction using conventional techniques and apparatus then removes the organic solvent. The resulting microspheres are washed, filtered and dried prior to combining them with an appropriate suspension medium in accordance with the present invention

Whatever production method is ultimately selected for production of the particulate compositions, the resulting powders have a number of advantageous properties that make them particularly compatible for use in devices for inhalation therapies. In particular, the physical characteristics of the particulate compositions make them extremely effective for use in dry powder inhalers. As such, the particulate compositions provide for the effective pulmonary administration of active agents.

In order to maximize dispersibility, dispersion stability and optimize distribution upon administration, the MMD of the particulate compositions is preferably about 0.5-50 μm, more preferably 1-20 μm and most preferably 0.5-5 μm. In especially preferred embodiments the mean MMD of the particulate compositions is less than 20 μm or less than 10 μm. More preferably the MMD is less than about 7 μm or 5 μm, and even more preferably less than about 2.5 μm. Other preferred embodiments will comprise preparations wherein the MMD of the particulate compositions is between about 1 μm and 5 μm. In especially preferred embodiments the particulate compositions will comprise a powder of dry, hollow, porous microspherical shells of approximately 1 to 10 μm or 1 to 5 μm in diameter, with shell thicknesses of approximately 0.1 μm to approximately 0.5 μm. It is a particular advantage of the present invention that the particulate concentration of the dispersions and structural matrix components can be adjusted to optimize the delivery characteristics of the selected particle size.

The foregoing description will be more fully understood with reference to the following Examples. Such Examples, are, however, merely representative of preferred methods of practicing the present invention and should not be read as limiting the scope of the invention.

### EXAMPLE 1

Preparation of Spray-Dried Budesonide Particles

Hollow porous budesonide particles were prepared by a two-step process. In the first step, 54 mg of budesonide (Vinchem, Chatham, N.J.), and 0.775 g of DSPC were dissolved in 2 ml of chloroform:methanol (2:1). The chloroform:methanol was then evaporated to obtain a thin film of the phospholipid/steroid mixture. The phospholipid/steroid mixture was then dispersed in 30.5 g of hot deionized water (T=60 to 70° C.) using an Ultra-Turrax mixer (model T-25) at 8000 rpm for 2 to 5 minutes. 12.8 g of perfluorooctyl ethane was then added dropwise during mixing. After the addition was complete, the emulsion was mixed for an additional period of not less than 4 minutes. The coarse emulsion was then passed through a high pressure homogenizer (Avestin, Ottawa, Canada) at 18,000 psi for 5 passes. The resulting submicron fluorocarbon-in-water with steroid solubilized in the lipid monolayer surrounding the droplets was utilized as the feedstock in for the second step, i.e. spray-drying on a B-191 Mini Spray-Drier (Buichi, Flawil, Switzerland). Calcium chloride (0 or 0.65 mg) was added in 2.5 g of water to the fluorocarbon-in-water emulsion immediately prior to spray drying. The following spray conditions were employed: aspiration=100%, inlet temperature=85° C., outlet temperature=60° C., feed pump=1.9 mL min⁻¹, atomizer pressure=60-65 psig, atomizer flow rate=30-35 cm. The aspiration flow (69-75%) was adjusted to maintain an exhaust bag pressure of 30-31 mbar. Free flowing white powders were collected using a standard cyclone separator.

The aerosol characteristics of the calcium containing formulation was examined in several passive dry powder inhaler devices (Eclipse® (Aventis), Turbospin® (PH&T), Cipla Rotahaler®, Glaxo Rotahaler®, and Hovione FlowCaps®). The emitted dose was determined gravimetrically at comfortable inhalation flow rate (peak flow rate=20-62 L/min depending on the resistance of the device), and at a forced inhalation flow rate (peak flow rate 37-90 L/min). Under comfortable inhalation flow conditions the range of emitted doses was between 89 and 96% with a mean emitted dose of 94%. Under forced inhalation flow, the emitted dose varied between 94 and 103%, with a mean emitted dose of 99%. The fact that multiple devices with high and low resistance are able to effectively disperse the powders more or less independent of inspiratory flow rate speaks volumes to the flowability of the budesonide powder tested. Table 1 depicts the results.

TABLE 1
Budesonide Aerosol Characteristics

| | | Peak | | | | |
|---|---|---|---|---|---|---|
| | | Flow | Emitted | | | FPD |
| | | Rate | Dose | Total | MMAD | (% < |
| Device | Resistance | (LPM) | (%) | Dose | (um) | 3.3 um) |
| Eclipse | 0.19 | 20 | 89 | 72 | 3.2 | 37 |
| | | | | 68 | 3.0 | 36 |
| | | 41 | 94 | 73 | 2.0 | 50 |
| | | | | 73 | 2.3 | 44 |
| Flowcaps | 0.15-.20 | 23 | 93 | 66 | 4.3 | 21 |
| | | | | 46 | 3.7 | 17 |
| | | 37 | 102 | 70 | 2.6 | 40 |
| | | | | 72 | 3.0 | 36 |
| Cipla | 0.16 | 23 | 96 | 76 | 4.0 | 28 |
| Rotohaler | | | | 78 | 3.9 | 29 |
| | | 43 | 98 | 74 | 2.9 | 42 |
| | | | | 86 | 2.9 | 47 |
| Turbospin | 0.09 | 24 | 95 | 87 | 3.5 | 37 |
| | | | | 93 | 3.6 | 40 |
| | | 60 | 96 | 77 | 2.4 | 46 |
| | | | | 73 | 2.3 | 44 |
| Glaxo | 0.04 | 62 | 95 | 73 | 3.1 | 36 |
| Rotohaler | | | | 72 | 3.1 | 36 |
| | | 90 | 103 | 88 | 3.2 | 49 |
| | | | | 85 | 3.3 | 49 |

### EXAMPLE 2

Lung deposition using a DPI at two different peak inspiratory flow rates of budesonide powders was investigated. Plasma pharmacokinetics at the two different flow rates was compared to those obtained from the Pulmicort Turbuhaler® (Astra Zeneca, Lund, Sweden).

Spray-dried budesonide particles were prepared by first dispersing micronized budesonide crystals in water with the aid of DSPC under shear. The oil phase was then added dropwise to create a complex dispersion comprised of emulsion droplets and small budesonide crystals. The resulting dispersion was then spray-dried under conditions similar to Example 1. The budesonide concentration was 5.0% w/w in the powder.

The PS_{bud} powder was radiolabeled with ^{99m}Tc and deposition determined by gamma scintigraphy. In-vitro experiments confirmed radiolabel acted as a valid marker for drug. Charcoal was administered orally to reduce extra-pulmonary absorption of budesonide, and plasma budesonide concentrations were measured for 12 hour after all treatments. Eight healthy subjects completed the following 3 treatments in a cross-over study:

1. Eclipse Low flow:

PS_{bud} (0.37 mg budesonide) inhaled from the Eclipse DPI at a peak inspiratory flow (PIF) of 29 L/min (SD=3.6)

2. Eclipse High Flow:

PS_{bud} (0.37 mg budesonide) inhaled from the Eclipse DPI at a PIF of 44 L/min (SD=4.2)

3. Turbuhaler:

0.8 mg budesonide inhaled from Turbuhaler at 60 L/min

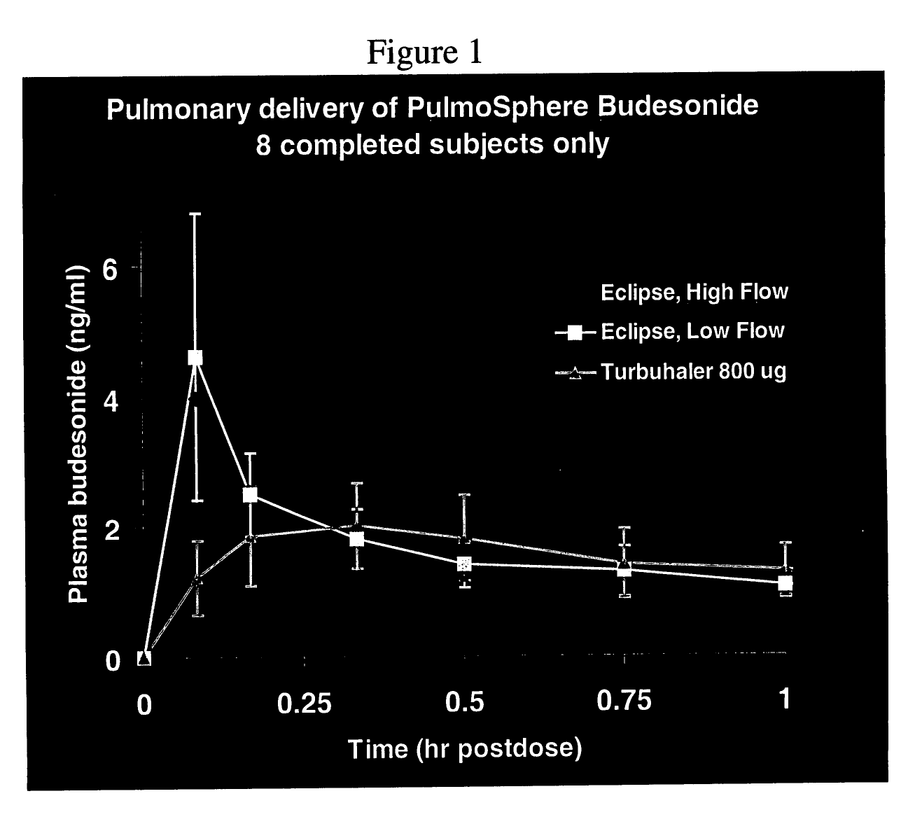

Results of the pharmokinetic study are depicted in FIG. 1 and summarized in Table 2. Interpatient variability in lung deposition expressed as the relative standard deviation in % about the mean lung deposition value is summarized in Table 3.

TABLE 2
Budesonide pK Summary

| Formulation | Flow rate | Deposition | Mean Tₘₐₓ |
|---|---|---|---|
| PS_{bud} | 29 | 57 ± 7 | 0.08 |
| PS_{bud} | 44 | 58 ± 8 | 0.10 |
| TurbuHaler | 35 | 15 | n.a. |
| TurbuHaler | 60* | 28* | .28 |

*From Borgstrom, L. et al. “Lung deposition of budesonide inhaled via Turbohaler ®: a comparison with terbutaline sulphate in normal subjects” Eur Respir. J. 1994, 7, 69-73.

TABLE 3
Interpatient Variability in Lung Deposition

| | | Interpatient Variability in |
|---|---|---|
| Formulation/Device | Q (LPM) | Lung Deposition (RSD, %) |
| Pulmicort Turbuhaler | 58 | 34 |
| n = 10 | 36 | 22 |
| PulmoSphere Eclipse | 44 | 13 |
| n = 8 | 29 | 11 |

The interpatient variability is significantly reduced for PulmoSphere formulation relative to the Pulmicort Turbuhaler.

### EXAMPLE 3

Leuprolide Acetate Particles

A single feed solution is prepared under defined conditions. The feed solution is comprised of leuprolide acetate in the aqueous phase of a fluorocarbon-in-water emulsion. The emulsion composition is listed in Table 3 below. Accordingly, DSPC and calcium chloride dihydrate are dispersed in approximately 400 mL SWFI (T=60-70 C) using an Ultra-Turrax T-50 mixer at 800 rpm for 2 to 5 minutes. The perflubron is then added drop wise during mixing. After the addition is complete, the emulsion is mixed for an additional period of not less than 5 minutes at 10,000 rpm. The resulting coarse emulsion is then homogenized under high pressure with an Avestin C-5 homogenizer (Ottawa, Canada) at 19,000 psi for 5 discrete passes. The emulsion is transferred to the Potent Molecule Laboratory for Leuprolide Acetate addition and spray drying.

TABLE 3
Leuprolide Acetate Emulsion Composition

| Emulsion Components | Amount (grams) | % solids |
|---|---|---|
| DSPC | 7.33 | 73% |
| Calcium Chloride | 0.67 | 7% |
| Perflubron | 200 | NA |
| SWFI | 400 | NA |
| Leuprolide Acetate | 2.00 | 20% |

Aerosol Data:

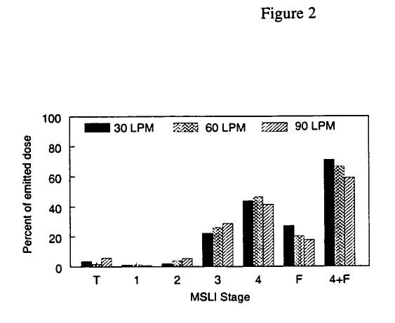

Deposition analysis is performed using a multi-stage liquid impinger (MSLI). The apparatus consists of four concurrent stages and a terminal filter, each containing an aliquot of appropriate solvent for Leuprolide Acetate analysis. The powder was administered by inhalation as a dry powder through the Turbospin device (PH&T) at 30, 60, and 90 LPM. The aerosol performance is described below in Table 4, and the MSLI deposition is profiled in FIG. 2. Only a minor dependence of the deposition is observed across a wide range of flow rate.

TABLE 4
Flow rate dependence of aerosol properties for a leuprolide
Pulmosphere formulation delivered from the Turbospin
DPI device

| Q (LPM) | MMAD (μm) | FPF_{4+F} (%) |
|---|---|---|
| 30 | 3.3 | 71 |
| 60 | 2.4 | 70 |
| 90 | 2.0 | 63 |

Little difference is noted in FPF_{4+F} as a function of flow rate, indicating that little dependence in lung deposition would be expected as a function of flow rate.

### EXAMPLE 4

Flow Rate Independent Deposition for Budesonide PulmoSphere Formulations from the Hovione Flowcaps Passive DPI Device

The budesonide PulmoSphere formulation of Example 2 was tested in the MSLI following dispersion from the Hovione Flowcaps passive DPI. The results are presented in Table 5.

TABLE 5

| Q (LPM) | MMAD (μm) | FPF_{4+F} (%) |
|---|---|---|
| 45 | 3.2 | 69 |
| 25 | 2.8 | 66 |

Little difference is noted in FPF_{4+F} as a function of flow rate under either profiles corresponding to forceful and comfortable inhalation, respectively. Hence, this formulation would be expected to show lung deposition independent of inspiratory flow rate.

### EXAMPLE 5

Tobramycin particles were manufactured using the same general procedure set forth in Example 1. The particles were ^{99m}Tc radiolabeled. 12 volunteers completed a five-period crossover study. To identify whole lung distribution via scintigraphy, subjects inhaled a single labeled capsule containing 25 mg of tobramycin formulation on three separate occasions at a flow rate of 72 LPM. Subjects next inhaled ^{99m}Tc TOBI® (Pathogeneses Corp) 5 ml/300 mg. Deposition and blood samples were obtained. At the final visit, six 25 mg doses of unlabeled formulation were inhaled and blood samples taken. Mean whole lung deposition was 34±5% and TOBI 5±2% (50% of dose nebulized). Serum Cmax values were 0.6 μg/ml for the tobramycin formulation according to the invention and 0.28 pg/ml with TOBI. The actual dose was 54% of TOBI and plasma tobramycin AUC was more than double TOBI (4.4 vs. 2.1 μg hr/ml). Intrasubject dose variability did not exceed 6%. The interpatient variability is summarized in Table 6.

TABLE 6

| | | Interpatient Variability |
|---|---|---|
| Formulation/Device | Q (LPM) | In Lung Deposition (RSD, %) |
| Nebulized Tobi | Tidal breathing | 40 |
| PulmoSphere Turbospin | 60 | 17 |

The interpatient variability is significantly reduced for PulmoSphere formulation relative to the nebulized Tobi formulation.

The invention has now been described in detail for purposes of clarity and understanding. However, it will be appreciated that certain changes and modifications may be practiced within the scope of the appended claims.

We claim:

1. A method for inhalation of a dry powder drug comprising:
providing a dry powder drug composition comprising particles comprising a lipid matrix and a particle size of 1-30 microns, mass median aerodynamic diameter of less than 5 microns, and bulk density of less than 0.5 g/cm³;
loading the composition into a passive dry powder inhaler; and
inhaling the drug composition from the inhaler resulting in an emitted dose substantially independent of device resistance and lung deposition substantially independent of inhalation flow rate.

2. A method according to claim 1 wherein the emitted dose is at least 60%.

3. A method according to claim 2 comprising an emitted dose of at least 80%.

4. A method according to claim 1 comprising a FPF_{4+F} of at least 60%.

5. A method according to claim 1 wherein the lipid comprises a phospholipid selected from the group consisting of dipalmitoylphosphatidylcholine, disteroylphosphatidylcholine, diarachidoylphosphatidylcholine dibehenoylphosphatidylcholine, diphosphatidyl glycerol, short-chain phosphatidylcholines, long-chain saturated phosphatidylethanolamines, long-chain saturated phosphatidylserines, long-chain saturated phosphatidylglycerols, and long-chain saturated phosphatidylinositols.

6. A method according to claim 1 wherein the inhaler comprises a resistance of less than 0.60 (cmH₂O)^{1/2}/L min⁻¹.

7. A method according to claim 6 wherein the inhaler comprises a resistance within the range of 0.01-0.30 (cmH₂O)^{1/2}/Lmin⁻¹

8. A method of claim 1 wherein the inhalation flow rate is less than about 90 L/min.

9. A method of claim 8 wherein the inhalation flow rate is within the range of about 10-60 L/min.

10. A method of claim 9 wherein the inhalation flow rate is within the range of 12-45 L/min.

11. A method of claim 1 wherein the lung deposition is greater than 25%.

12. A method according to claim 1 wherein the lung deposition is greater than 30%.

13. A method according to claim 1 wherein the lung deposition is greater than 50%.

14. A method according to claim 1 wherein the drug is selected from the group consisting of budesonide, tobramycin sulfate, leuprolide acetate, Amphotericin B, and PTH.

15. A method of claim 1 wherein the powder comprises hollow porous microparticles.

16. A method for inhalation of a dry powder drug comprising:
providing a dry powder drug composition comprising a hydrophobic active agent, said composition comprising particles comprising a lipid matrix and a particle size of 1-30 microns, mass median aerodynamic diameter of less than 5 microns, and bulk density of less than 0.5 g/cm³;
loading the composition into a passive dry powder inhaler;
inhaling the drug composition from the inhaler in order to achieve a Tmax within 15 minutes of the inhalation.

17. A method according to claim 16 wherein the active agent is amphotericin B.

18. A method according to claim 16 wherein the active agent is budesonide.

19. A method according to claim 18 wherein T max is achieved within 10 minutes of the inhalation.

20. A method according to claim 16 wherein the lipid comprises a phospholipid selected from the group consisting of dipalmitoylphosphatidylcholine, disteroylphosphatidylcholine, diarachidoylphosphatidylcholine dibehenoylphosphatidylcholine, diphosphatidyl glycerol, short-chain phosphatidylcholines, long-chain saturated phosphatidylethanolamines, long-chain saturated phosphatidylserines, long-chain saturated phosphatidylglycerols, and long-chain saturated phosphatidylinositols.
